(19) United States
(12) Patent Application Publication
(10) Pub. No.: US 2025/0134789 A1
(43) Pub. Date: May 1, 2025

(54) COMPOSITION COMPRISING CYSTEINE, A PARTICULAR FATTY ACID TRIGLYCERIDE AND AN ADDITIONAL REDUCING AGENT

(71) Applicant: L'OREAL, Paris (FR)

(72) Inventors: Simon DONCK, JOHANNESBURG (ZA); Kwezikazi MOLAMODI, JOHANNESBURG (ZA); Shannon ATTWELL, JOHANNESBURG (ZA); Jason EASON, JOHANNESBURG (ZA)

(21) Appl. No.: 18/292,246

(22) PCT Filed: Jul. 26, 2022
(86) PCT No.: PCT/EP2022/070863

§ 371 Date: Jan. 25, 2024

(30) Foreign Application Priority Data
Jul. 27, 2021 (ZA) 2021/05298
Sep. 22, 2021 (FR) 2109991
Nov. 9, 2021 (ZA) 2021/08808
Dec. 10, 2021 (FR) 2113266

(51) Int. Cl. A61K 8/46; A61K 8/37; A61K 8/41; A61Q 5/06

(52) U.S. Cl. CPC A61K 8/46; A61K 8/375; A61K 8/416; A61Q 5/06

(57) ABSTRACT

The present invention relates to a composition useful for styling and/or conditioning keratin fibers, and in particular human keratin fibers such as the hair, which comprises cysteine and/or one of its derivatives, a particular fatty acid triglyceride and optionally an additional reducing agent. The invention also relates to a cosmetic process for styling and/or conditioning keratin fibres using such a composition.

The present invention relates to a composition useful for styling and/or conditioning keratin fibers, and in particular human keratin fibers such as the hair, which comprises cysteine and/or one of its derivatives, a particular fatty acid triglyceride and optionally an additional reducing agent.

The invention also relates to a cosmetic process for styling and/or conditioning keratin fibres using such a composition.

Many people are unsatisfied with the way their hair looks, and have difficulty in styling it. Curly hair is particularly difficult to comb and detangle, requiring a lot of time and effort for a result that is most of the time disappointing.

Moreover, hair is generally damaged and embrittled by the action of external atmospheric agents such as light and bad weather, but also by mechanical or chemical treatments, such as brushing, combing, dyeing, bleaching, permanent-waving and/or relaxing. Hair is thus damaged by these various factors and may over time become dry, coarse or dull, especially in fragile areas.

Thus, to overcome these drawbacks, it is common practice to use care compositions that condition the hair appropriately, giving it satisfactory conditioning properties, especially in terms of smoothness, sheen, softness, suppleness, lightness, a natural feel and good disentangling properties.

These hair care compositions may be, for example, conditioning shampoos, hair conditioners, masks or sera, and may be in the form of gels, hair lotions or care creams that are more or less thick. These compositions may be rinsed-out or leave-in compositions.

These compositions generally comprise a combination of cationic conditioning agents such as cationic surfactants, cationic polymers, silicones and/or fatty substances, such as fatty alcohols, in order to give the hair satisfactory cosmetic properties, especially in terms of softness, smoothness and suppleness.

However, such conditioning agents, and in particular fatty alcohols, often have the drawback of making the hair very lank and heavy, which leads to the phenomenon commonly known as the “dull effect”. In other words, fatty alcohols become deposited in a large amount on the hair, which has the consequence of making the hair lank, oily and dull.

Nevertheless, removal or reduction of these fatty materials decreases the conditioning effects, and thus the conditioning properties of the hair.

Most of these compositions also comprise silicones. Indeed, these compounds are known to improve the cosmetic properties of hair, especially in terms of smoothness and flexibility (as shown in JP 48 (1973)-19941, JP 56 (1986)-92808, and U.S. Pat. No. 5,374,421). However, several disadvantages remain. For instance, hair treated with these compositions remains lank and sticky, and may present a poor combability.

Therefore, there is a real need to develop compositions that do not have the drawbacks described above, i.e. which are able to improve the conditioning properties of the hair, especially in terms of wet and dry combability, suppleness, smoothness, manageability and shine, without overloading the hair, or leading to a greasy feel or appearance.

The compositions shall also improve the combing, styling and detangling properties of the hair, in particular curly hair to kinky hair while reducing knots and avoiding breakage or damage of the fibers.

It has now been discovered that a composition, comprising cysteine and/or one of its derivatives in combination with a particular fatty acid triglyceride, makes it possible to achieve the objectives outlined above. In particular, it has been found that hair treated with such a combination is easier to comb and detangle and presents better conditioning properties, particularly in terms of smoothness, suppleness and shine. The hair is also more manageable leading thus to a better styling, and presents less breakage. These benefits are also lasting.

Thus, the subject of the invention is especially a composition comprising:
(i) cysteine and/or one of its derivatives such as N-alkanoyl (C1-C12) cysteine and cysteine alkyl (C1-C12) esters and salts thereof, in a total amount of lower than or equal to 1% by weight, relative to the total weight of the composition,
(ii) one or more fatty acid triglyceride(s) containing from 6 to 16 carbon atoms in a total amount of more than or equal to 1% by weight, relative to the total weight of the composition, and
(iii) optionally one or more additional reducing agents other than cysteine and/or one of its derivatives (i) in a total amount of lower than or equal to 1% by weight, relative to the total weight of the composition;
the pH of said composition ranging from 7.5 to 10.

The composition of the present invention is able to improve the conditioning properties of keratin fibres, in particular human keratin fibres such as the hair, especially in terms of shine, softness, smoothness and suppleness while at same time giving the hair a good wet and dry combability, as well as an improved manageability.

The hair, notably curly hair, thus treated with composition of the present invention is easier to style and allows the consumer to obtain the desired hairstyle.

The composition of the present invention also respects the integrity of the fibres and provides a better moisturizing effect. The hair is also healthier and stronger, allowing it to better resist to combing and detangling.

The invention also relates to a cosmetic process for styling and/or conditioning keratin fibres, in particular human keratin fibres such as hair, which consists in applying to said keratin fibres a composition as previously defined, and after an optional leave-on time, optionally removing it by rinsing.

Other subjects, characteristics, aspects and advantages of the invention will emerge even more clearly on reading the description and the example that follows.

In that which follows and unless otherwise indicated, the limits of a range of values are included within this range, in particular in the expressions “of between” and “ranging from . . . to . . . “.

Moreover, the expression “at least one” used in the present description is equivalent to the expression “one or more”.

### (i) Cysteine

The composition according to the present invention comprises cysteine and/or one of its derivatives such as N-alkanoyl (C1-C12) cysteine and cysteine alkyl (C1-C12) esters and salts thereof, in a total amount of lower than or equal to 1% by weight, relative to the total weight of the composition.

The total amount of cysteine and/or one of its derivatives such as N-alkanoyl (C1-C12) cysteine and cysteine alkyl (C1-C12) esters and salts thereof, present in the composition according to the present invention, preferably ranges from 0.05 to 1% by weight, more preferentially from 0.1 to 1% by weight, and better still from 0.5 to 0.8% by weight, relative to the total weight of the composition.

### (ii) Fatty Acid Triglyceride

The composition according to the present invention further comprises one or more fatty acid triglyceride(s) (ii) containing from 6 to 16 carbon atoms.

In particular, the fatty acid triglycerides (ii) of the invention have the following general formula:

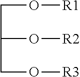

Wherein, R1, R2 and R3, which may be identical or different, denote linear or branched, saturated or unsaturated C6-C16 alkyl groups, preferably C6-C14 alkyl groups and more preferably C6-C12 alkyl groups.

The fatty acid triglyceride(s) useful according to the present invention may be from triglycerides of plant origin, such as plant oils, or from triglycerides of synthetic origin.

Preferably, the fatty acid triglyceride(s) contain from 6 to 14 carbon atoms, and more preferentially from 6 to 12 carbon atoms.

Preferably, plant oils which can be used according to the invention comprise more than 50% by weight of fatty acid triglyceride(s) containing from 6 to to 16 carbon atoms, preferably from 6 to 14, more preferentially from 6 to 12 carbon atoms. Advantageously the fatty acid triglyceride(s) are chosen from coconut oil, caprylic/capric acid triglycerides, palm kernel oil, babasu oil, cuphea oil and mixtures thereof.

The total amount of fatty acid triglyceride(s) (ii) containing from 6 to 16 carbon atoms, that are present in the composition according to the present invention, is greater than or equal to 1% by weight, relative to the total weight of the composition. Preferably, the total amount of fatty acid triglyceride(s) (ii) containing from 6 to 16 carbon atoms ranges from 1 to 15% by weight, and more preferentially from 2 to 10% by weight, relative to the total weight of the composition.

The weight ratio (R) between the total amount of fatty acid triglyceride(s) (ii) containing from 6 to 16 and the total amount of cysteine and/or one of its derivatives (i), present in the composition according to the present invention, is preferably greater than or equal to 1. More preferentially, this weight ratio (R) ranges from 1 to 20, and better still from 2 to 15.

(iii) Additional Reducing Agents

The composition according to the present invention optionally comprises one or more additional reducing agents other than cysteine and/or one of its derivatives (i) in a total amount of lower than or equal to 1% by weight, relative to the total weight of the composition. In other words, the total amount of the additional reducing agents (iii) other than cysteine and/or one of its derivatives (i), when they are present in the composition of the invention, is lower than or equal to 1% by weight, relative to the total weight of the composition

The additional reducing agents (iii) other than cysteine and/or one of its derivatives (i), are preferably chosen from thiol reducing agents, different from cysteine or its derivatives (i), non-thiol reducing agents and mixtures thereof.

The expression “thiol reducing agent” means a reducing agent containing at least one thiol group. Examples of thiol reducing agent(s) different from cysteine or its derivatives (i) correspond to thioglycolic acid and derivatives thereof, in particular esters thereof such as glycerol or glycol monothioglycolate; thiolactic acid and derivatives thereof, in particular esters thereof such as glycerol monothiolactate; 3-mercaptopropionic acid and derivatives thereof, in particular esters thereof such as glycerol 3-mercaptopropionate and ethyleneglycol 3-mercaptopropionate; cysteamine and derivatives thereof, in particular C₁-C₄ acyl derivatives thereof such as N-acetylcysteamine and N-propionylcysteamine; mono-thioglycerol and derivatives thereof, in particular esters; and salts thereof.

The expression “non-thiol reducing agent” means a reducing agent which does not contain any thiol group. Examples of non-thiol reducing agent(s) correspond to sulfites, sulfite derivatives, sulfinates, phosphines, sugars, dihydroxybenzene derivatives, reductones, hydrides, ascorbic acid, ascorbic acid derivatives, tocopherols, tocopherol derivatives, EDTA, panthenol, selenium sulfide, zinc formosulfoxylate and mixtures thereof.

The additional reducing agents (iii) other than cysteine and/or one of its derivatives (i), are preferably chosen from non-thiol reducing agents and mixtures thereof, more preferentially from sulfites, sulfite derivatives, ascorbic acid, ascorbic acid derivatives, tocopherols, tocopherol derivatives and mixtures thereof.

The term “sulfite derivatives” essentially denotes bisulfites and sulfite diesters of formula R—O—SO₂—R′, with R and R′ denoting C₁-C₁₀ alkyl groups.

The term “ascorbic acid derivatives” essentially denotes erythorbic acid, isoascorbic acid, magnesium ascorbyl phosphate and ascorbyl glucoside.

The tocopherols that can be used in the present invention include for example alpha tocopherol, beta tocopherol, gamma tocopherol and delta tocopherol, while the term “tocopherol derivatives” essentially denotes the esters of tocopherols, such as tocopheryl acetate.

The additional reducing agents (iii) may be used especially in the form of salts, in particular alkali metal salts such as sodium and potassium salts, alkaline-earth metal salts, for example magnesium and calcium salts, ammonium salts, amine salts and amino alcohol salts.

In a particularly preferred manner, the additional reducing agents (iii) other than cysteine and/or one of its derivatives (i), are chosen from alkali metal sulfites, alkali metal bisulfites, precursors of these sulfites or bisulfites, and mixtures thereof. More preferentially, the additional reducing agents (iii) are chosen from sodium sulfite, sodium bisulfite, sodium metabisulfite and mixtures thereof.

According to another particularly preferred embodiment, the additional reducing agents (iii) other than cysteine and/or one of its derivatives (i) are chosen from sulfites, sulfite derivatives, ascorbic acid, ascorbic acid derivatives, tocopherols, tocopherol derivatives and mixtures thereof. More preferably, the additional reducing agents (iii) according to this embodiment, correspond to a mixture of at least two distinct non-thiol reducing agents, wherein preferably:
at least one is chosen from sulfites, sulfite derivatives and mixtures thereof, more preferentially from alkali metal sulfites, alkali metal bisulfites, precursors of these sulfites or bisulfites, and mixtures thereof, and better still from sodium sulfite, sodium bisulfite, sodium metabisulfite and mixtures thereof, and
the other one is chosen from ascorbic acid, ascorbic acid derivatives, and mixtures thereof, and more preferentially from ascorbic acid, alpha-tocopherol, tocopheryl acetate, erythorbic acid, isoascorbic acid, magnesium ascorbyl phosphate, ascorbyl glucoside and mixtures thereof.

The total amount of the additional reducing agents (iii) other than cysteine and/or one of its derivatives (i), when they are present in the composition according to the invention, is lower than or equal to 1% by weight, preferably ranges from 0.05 to 1% by weight, more preferentially from 0.1 to 0.8% by weight, better still from 0.1 to 0.5% by weight, and even better from 0.15 to 0.3% by weight, relative to the total weight of the composition.

In a preferred embodiment, the additional reducing agents (iii) other than cysteine and/or one of its derivatives (i), are chosen from sulfites, sulfite derivatives, ascorbic acid, ascorbic acid derivatives and mixtures thereof, and the total amount of sulfites, sulfite derivatives, ascorbic acid and ascorbic acid derivatives, present in the composition according to the invention, is lower than or equal to 1% by weight, preferably ranges from 0.05 to 1% by weight, more preferentially from 0.1 to 0.8% by weight, better still from 0.1 to 0.5% by weight, and even better from 0.15 to 0.3% by weight, relative to the total weight of the composition.

The weight ratio (Ra) between the total amount of cysteine and/or one of its derivatives (i) and the total amount of the additional reducing agents (iii) other than cysteine and/or one of its derivatives (i), when they are present in the composition according to the present invention, is preferably greater than or equal to 1. More preferentially, this weight ratio (Ra) ranges from 1 to 15, and better still from 2 to 10.

In a particular embodiment of the invention, the composition comprises one or more additional reducing agents (iii) other than cysteine and/or one of its derivatives (i), as described above.

In another particular embodiment of the invention, the composition is free of additional reducing agent other than cysteine and/or one of its derivatives (i).

Thus, according to this particular embodiment, the composition comprises:
(i) cysteine and/or one of its derivatives such as N-alkanoyl (C1-C12) cysteine and cysteine alkyl (C1-C12) esters and salts thereof in a total amount of lower than or equal to 1% by weight, relative to the total weight of the composition,
(ii) one or more fatty acid triglyceride(s) containing from 6 to 16 carbon atoms in a total amount of more than or equal to 1% by weight, relative to the total weight of the composition;
the pH of said composition ranging from 7.5 to 10, and
said composition being free of additional reducing agent other than cysteine and/or one of its derivatives (i).

For the purposes of the present invention, the term “free of” refers to a composition which does not contain (0%) additional reducing agent other than cysteine or its derivatives (i) or which contains less than 0.1% by weight of additional reducing agents, relative to the total weight of the composition.

### Additional Fatty Substances

The composition according to the present invention may further comprise one or more additional fatty substance(s), different from the fatty acid triglycerides (ii) previously described.

The term “fatty substance” means an organic compound that is insoluble in water at ordinary temperature (25° C.) and at atmospheric pressure (760 mmHg) (solubility of less than 5%, preferably less than 1% and even more preferentially less than 0.1%). They bear in their structure at least one hydrocarbon-based chain including at least 6 carbon atoms or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

Preferably, the fatty substances of the invention do not contain any salified or unsalified carboxylic acid groups (—C(O) OH or —C(O)O⁻). Particularly, the fatty substances of the invention are neither polyoxyalkylenated nor polyglycerolated.

The term “non-silicone fatty substance” means a fatty substance not containing any silicon atoms (Si) and the term “silicone fatty substance” means a fatty substance containing at least one silicon atom.

More particularly, the additional fatty substance(s), different from the fatty acid triglyceride(s) containing from 6 to 16 carbon atoms, are chosen from C₆ to C₁₆ hydrocarbons, hydrocarbons containing more than 16 carbon atoms, non-silicone oils of animal origin, triglycerides different from the fatty acid triglycerides (ii) previously described, fluoro oils, non polyoxyalkylenated fatty alcohols, esters of fatty acids and/or of fatty alcohols other than triglycerides, plant waxes, non-silicone waxes, silicones and mixtures thereof.

It is recalled that, for the purposes of the invention, the fatty alcohols, fatty esters and fatty acids more particularly contain one or more linear or branched, saturated or unsaturated hydrocarbon-based groups comprising 6 to 30 carbon atoms, which are optionally substituted, in particular, with one or more (in particular 1 to 4) hydroxyl groups. If they are unsaturated, these compounds may comprise one to three conjugated or unconjugated carbon-carbon double bonds.

As regards the C₆ to C₁₆ hydrocarbons, they are linear, branched or optionally cyclic, and are preferably alkanes. Examples that may be mentioned include hexane, dodecane and isoparaffins such as isohexadecane and isodecane.

A hydrocarbon-based oil of animal origin that may be mentioned is perhydrosqualene.

The triglyceride different from the fatty acid triglyceride (ii) containing from 6 to 16 carbon atoms are preferably chosen from liquid fatty acid triglycerides containing from 18 to 30 carbon atoms, for instance sunflower oil, corn oil, soybean oil, pumpkin oil, rapeseed oil, grapeseed oil, sesame oil, hazelnut oil, apricot oil, macadamia oil, arara oil, castor oil, avocado oil, jojoba oil, and mixtures thereof.

The linear or branched hydrocarbons of mineral or synthetic origin, containing more than 16 carbon atoms, are preferably chosen from liquid paraffins, petroleum jelly, liquid petroleum jelly, polydecenes and hydrogenated polyisobutene such as Parleam®.

The fluoro oils may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or alternatively bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The non polyoxyalkylenated fatty alcohols that may be used in the composition according to the invention are saturated or unsaturated, and linear or branched, and include from 8 to 30 carbon atoms and more particularly from 10 to 24 carbon atoms. Examples that may be mentioned include cetyl alcohol, stearyl alcohol and the mixture thereof (cetylstearyl alcohol), octyldodecanol, 2-butyloctanol, 2-hexyldecanol, 2-undecylpentadecanol, oleyl alcohol and linoleyl alcohol.

The wax(es) that may be used in the composition according to the invention are chosen especially from carnauba wax, candelilla wax, esparto grass wax, paraffin wax, ozokerite, plant waxes, for instance olive wax, rice wax, hydrogenated jojoba wax or the absolute waxes of flowers such as the essential wax of blackcurrant blossom sold by the company Bertin (France), animal waxes, for instance beeswaxes, or modified beeswaxes (cerabellina); other waxes or waxy starting materials that may be used according to the invention are especially marine waxes such as the product sold by the company Sophim under the reference M82, and polyethylene waxes or polyolefin waxes in general.

As regards the fatty acid and/or fatty alcohol esters, which are advantageously different from the triglycerides mentioned above, mention may be made in particular of esters of saturated or unsaturated, linear or branched C₁ to C₂₆ aliphatic mono- or polyacids and of saturated or unsaturated, linear or branched C₁ to C₂₆ aliphatic mono- or polyalcohols, the total carbon number of the esters more particularly being greater than or equal to 10.

Among the monoesters, mention may be made of dihydroabietyl behenate; octyldodecyl behenate; isocetyl behenate; cetyl lactate; C12-C15 alkyl lactate; isostearyl lactate; lauryl lactate; linoleyl lactate; oleyl lactate; (iso) stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; myristyl stearate; octyl isononanoate; 2-ethylhexyl isononanoate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C₄ to C₂₂ dicarboxylic or tricarboxylic acids and of C₁ to C₂₂ alcohols and esters of mono-, di- or tricarboxylic acids and of C₂ to C₂₆ di-, tri-, tetra- or pentahydroxy alcohols may also be used.

Mention may be made especially of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, it is preferred to use ethyl, isopropyl, myristyl, cetyl or stearyl palmitate, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The composition may also comprise, as fatty ester, sugar esters and diesters of C₆ to C₃₀ and preferably C₁₂ to C₂₂ fatty acids. It is recalled that the term “sugar” means oxygenated hydrocarbon-based compounds that contain several alcohol functions, with or without aldehyde or ketone functions, and that include at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, in particular alkyl derivatives, such as methyl derivatives, for instance methylglucose.

The sugar esters of fatty acids may be chosen in particular from the group comprising the esters or mixtures of esters of sugars described previously and of linear or branched, saturated or unsaturated C₆ to C₃₀ and preferably C₁₂ to C₂₂ fatty acids. If they are unsaturated, these compounds may comprise one to three conjugated or unconjugated carbon-carbon double bonds.

The esters according to this alternative form can also be chosen from mono-, di-, tri- and tetraesters and polyesters, and mixtures thereof.

These esters may be, for example, oleates, laurates, palmitates, myristates, behenates, cocoates, stearates, linoleates, linolenates, caprates or arachidonates, or mixtures thereof such as, in particular, oleate/palmitate, oleate/stearate or palmitate/stearate mixed esters.

More particularly, use is made of mono- and diesters and in particular mono- or di-oleate-stearate-behenate-oleate/palmitate-linoleate-linolenate or -oleate/stearate of sucrose, glucose or methylglucose.

Mention may be made, by way of example, of the product sold under the name Glucate® DO by the company Amerchol, which is a methylglucose dioleate.

Examples of esters or mixtures of sugar esters of fatty acid that may also be mentioned include:
the products sold as F160, F140, F110, F90, F70 and SL40 by Crodesta, respectively denoting sucrose palmitate/stearates formed of 73% monoester and 27% di- and triester, of 61% monoester and 39% di-, tri- and tetraester, of 52% monoester and 48% di-, tri- and tetraester, of 45% monoester and 55% di-, tri- and tetraester, and of 39% monoester and 61% di-, tri- and tetraester, and sucrose monolaurate;
the products sold as Ryoto Sugar Esters, for example referenced B370 and corresponding to sucrose behenate formed of 20% monoester and 80% diester, triester and polyester;
the sucrose mono-dipalmitate/stearate sold by the company Goldschmidt under the name Tegosoft® PSE.

The silicones that may be used in accordance with the invention may be in the form of oils, waxes, resins or gums.

Preferably, the silicone is chosen from polydialkylsiloxanes, in particular polydimethylsiloxanes (PDMSs), and organomodified polysiloxanes including at least one functional group chosen from amino groups, aryl groups and alkoxy groups.

Organopolysiloxanes are defined in greater detail in Walter Noll's “Chemistry and Technology of Silicones” (1968), Academic Press. They may be volatile or non-volatile.

When they are volatile, the silicones are more particularly chosen from those having a boiling point of between 60° C. and 260° C., and more particularly still from: (I) cyclic polydialkylsiloxanes including from 3 to 7 and preferably from 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, and Silbione® 70045 V5 by Rhodia, and mixtures thereof.

Mention may also be made of cyclocopolymers of the dimethylsiloxane/methylalkylsiloxane type, such as Volatile Silicone® FZ 3109 sold by the company Union Carbide, having formula:

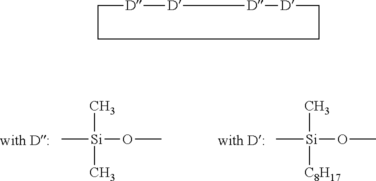

Mention may also be made of mixtures of cyclic polydialkylsiloxanes with organosilicon compounds, such as the mixture of octamethylcyclotetrasiloxane and tetra (trimethylsilyl) pentaerythritol (50/50) and the mixture of octamethylcyclotetrasiloxane and oxy-1,1′-bis(2,2,2′,2′,3,3′-hexatrimethylsilyloxy) neopentane;
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers, “Volatile Silicone Fluids for Cosmetics”.

Use is preferably made of non-volatile polydialkylsiloxanes, polydialkylsiloxane gums and resins, polyorganosiloxanes modified with the organofunctional groups above, and mixtures thereof.

These silicones are more particularly chosen from polydialkylsiloxanes, among which mention may be made mainly of polydimethylsiloxanes bearing trimethylsilyl end groups. The viscosity of the silicones is measured at 25° C. according to Standard ASTM 445 Appendix C.

Among these polydialkylsiloxanes, mention may be made, in a nonlimiting manner, of the following commercial products:
the Silbione oils of the 47 and 70 047 series or the Mirasil oils sold by Rhodia, for instance the oil 70 047 V 500 000,
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes having dimethylsilanol end groups known as dimethiconol (CTFA), such as the oils in the 48 series from Rhodia.

In this category of polydialkylsiloxanes, mention may also be made of the products sold under the names Abil Wax® 9800 and 9801 by the company Goldschmidt, which are poly (C₁-C₂₀) dialkylsiloxanes.

The silicone gums that can be used in accordance with the invention are in particular polydialkylsiloxanes and preferably polydimethylsiloxanes having high number-average molecular weights of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Products that may be used more particularly in accordance with the invention are mixtures such as:
the mixtures formed from a hydroxy-terminated polydimethylsiloxane or dimethiconol (CTFA), and from a cyclic polydimethylsiloxane, also known as cyclomethicone (CTFA), such as the product Q2 1401 sold by the company Dow Corning;
mixtures of a polydimethylsiloxane gum and a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;
mixtures of two PDMSs with different viscosities, and more particularly of a PDMS gum and of a PDMS oil, such as the product SF 1236 from the company General Electric. The product SF 1236 is a mixture of a gum SE 30 defined above, with a viscosity of 20 m²/s and of an oil SF 96 with a viscosity of 5×10⁻⁶ m²/s. This product preferably includes 15% of gum SE 30 and 85% of an oil SF 96. The organopolysiloxane resins that may be used in accordance with the invention are crosslinked siloxane systems containing the following units:
R₂SiO_{2/2}, R₃SiO_{1/2}, RSiO_{3/2} and SiO_{4/2},
in which R represents an alkyl containing 1 to 16 carbon atoms. Among these products, the ones that are particularly preferred are those in which R denotes a C₁-C₄ lower alkyl group, more particularly methyl.

Mention may be made, among these resins, of the product sold as Dow Corning 593 or those sold as Silicone Fluid SS 4230 and SS 4267 by General Electric, which are silicones having dimethyl/trimethylsiloxane structure.

Mention may also be made of the resins of the trimethylsiloxysilicate type, sold in particular as X22-4914, X21-5034 and X21-5037 by Shin-Etsu.

The organomodified silicones that may be used in accordance with the invention are silicones as defined previously and including in their structure one or more organofunctional groups attached via a hydrocarbon-based group.

The organomodified silicones may be polydiarylsiloxanes, in particular polydiphenylsiloxanes, and polyalkylarylsiloxanes functionalized with the organofunctional groups mentioned previously.

The polyalkylarylsiloxanes are particularly chosen from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

Among the organomodified silicones, mention may also be made of polyorganosiloxanes including:
substituted or unsubstituted amino groups, such as the products sold under the names GP 4 Silicone Fluid and GP 7100 by the company Genesee or the products sold under the names Q2 8220 and Dow Corning 929 or 939 by the company Dow Corning. The substituted amino groups are, in particular, C₁-C₄ aminoalkyl groups;
alkoxylated groups, such as the product sold under the name Silicone Copolymer F-755 by SWS Silicones and Abil Wax® 2428, 2434 and 2440 by the company Goldschmidt.

The silicones that can be used in the composition of the present invention may also be chosen from amino silicones.

The term “aminosilicone” denotes any silicone including at least one primary, secondary or tertiary amine or a quaternary ammonium group.

The weight-average molecular masses of these aminosilicones may be measured by gel permeation chromatography (GPC) at room temperature (25° C.), as polystyrene equivalent. The columns used are μ styragel columns. The eluent is THF and the flow rate is 1 ml/min. 200 μl of a 0.5% by weight solution of silicone in THF are injected. Detection is performed by refractometry and UV-metry.

Throughout the text hereinbelow, the term “silicone” is intended to denote, in accordance with what is generally accepted, any organosilicon polymer or oligomer of linear or cyclic, branched or crosslinked structure, of variable molecular weight, obtained by polymerization and/or polycondensation of suitably functionalized silanes, and formed essentially from a repetition of main units in which the silicon atoms are linked together via oxygen atoms (siloxane bond —Si—O—Si—), optionally substituted hydrocarbon-based groups being directly linked via a carbon atom to said silicon atoms. The hydrocarbon-based groups that are the most common are alkyl groups, notably C₁-C₁₀ alkyl groups and in particular methyl, fluoroalkyl groups, the alkyl part of which is C₁-C₁₀, and aryl groups and in particular phenyl.

The appropriate aminosilicones that may be used in accordance with the present invention comprise, without being limited thereto, volatile and non-volatile, cyclic, linear and branched aminosilicones, with a viscosity ranging from 5×10⁻⁶ to 2.5 m²/s at 25° C., for example from 1×10⁻⁵ to 1 m²/s.

Preferably, the aminosilicone(s) are chosen from:
a) the polysiloxanes corresponding to formula (I):

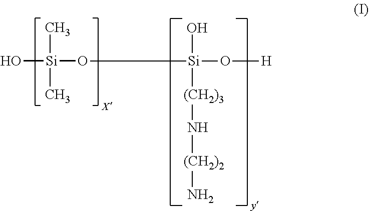

in which x′ and y′ are integers such that the weight-average molecular mass (Mw) is between 5000 and 500 000 g/mol;
b) the aminosilicones corresponding to formula (II):

R′ₐG₃₋ₐ-Si(OSiG₂)ₙ—(OSiG_{b}R′_{2-b})ₘ—O-SiG_{3-a′}-R′_{a′} (II)
in which:
   G, which may be identical or different, denotes a hydrogen atom or a phenyl, OH, C₁-C₈ alkyl, for example methyl, or C₁-C₈ alkoxy, for example methoxy, group,
   a and a′, which may be identical or different, denote 0 or an integer from 1 to 3, in particular 0, with the proviso that at least one from among a and a′ is equal to zero,
   b denotes 0 or 1, in particular 1,
   m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10; and
   R′, which may be identical or different, denotes a monovalent radical of formula —C_{q}H_{2q}L in which q is a number ranging from 2 to 8 and L is an optionally quaternized amino group chosen from the following groups:
      —NR″-Q-N(R″)₂,
      —N(R″)₂,
      —N⁺(R″)₃ A⁻,
      —N⁺H(R″)₂ A⁻,
      —N⁺H₂ (R″)A⁻,
      —NR″-Q-N⁺(R″)H₂ A⁻,
      —NR″-Q-N⁺(R″)₂H A⁻ and
      —NR″-Q-N⁺(R″)₃ A⁻,
in which R″, which may be identical or different, denotes hydrogen, phenyl, benzyl, or a saturated monovalent hydrocarbon-based radical, for example a C₁-C₂₀ alkyl radical; Q denotes a linear or branched group of formula CᵣH₂r, r being an integer ranging from 2 to 6, preferably from 2 to 4; and A represents a cosmetically acceptable anion, notably a halide such as fluoride, chloride, bromide or iodide.

According to a first embodiment, the aminosilicones corresponding to formula (II) are chosen from the silicones known as “trimethylsilyl amodimethicone”, corresponding to formula (III):

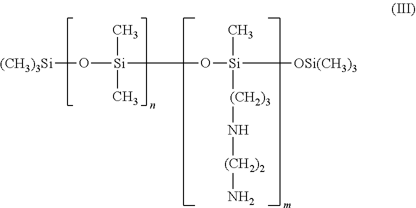

in which m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10.

According to a second embodiment, the aminosilicones corresponding to formula (II) are chosen from the silicones of formula (IV) below:

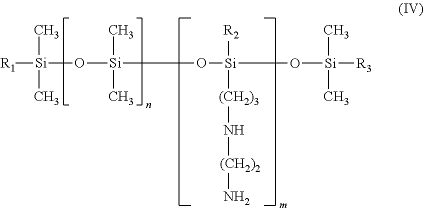

in which:
   m and n are numbers such that the sum (n+m) ranges from 1 to 1000, notably from 50 to 250 and more particularly from 100 to 200; n denoting a number from 0 to 999, notably from 49 to 249 and more particularly from 125 to 175, and m denoting a number from 1 to 1000, notably from 1 to 10 and more particularly from 1 to 5; and
   R1, R2 and R3, which may be identical or different, represent a hydroxyl or C₁-C₄ alkoxy radical, at least one of the radicals R₁ to R₃ denoting an alkoxy radical.

Preferably, the alkoxy radical is a methoxy radical.

The hydroxy/alkoxy mole ratio preferably ranges from 0.2:1 to 0.4:1, preferably from 0.25:1 to 0.35:1 and is more particularly equal to 0.3:1.

The weight-average molecular mass (Mw) of these silicones preferably ranges from 2000 to 1 000 000 g/mol and more particularly from 3500 to 200 000 g/mol.

According to a third embodiment, the aminosilicones corresponding to formula (II) are chosen from the silicones of formula (V) below:

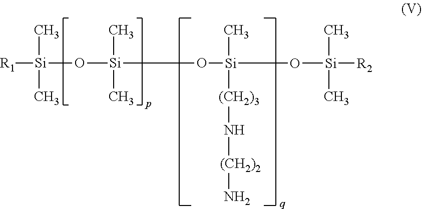

in which:
   p and q are numbers such that the sum (p+q) ranges from 1 to 1000, in particular from 50 to 350 and more particularly from 150 to 250; p denoting a number from 0 to 999, notably from 49 to 349 and more particularly from 159 to 239, and q denoting a number from 1 to 1000, notably from 1 to 10 and more particularly from 1 to 5; and
   R₁ and R₂, which are different, represent a hydroxyl or C₁-C₄ alkoxy radical, at least one of the radicals R₁ or R₂ denoting an alkoxy radical.

Preferably, the alkoxy radical is a methoxy radical.

The hydroxy/alkoxy mole ratio generally ranges from 1:0.8 to 1:1.1, preferably from 1:0.9 to 1:1 and more particularly is equal to 1:0.95.

The weight-average molecular mass (Mw) of the silicone preferably ranges from 2000 to 200 000 g/mol, more preferentially from 5000 to 100 000 g/mol and in particular from 10 000 to 50 000 g/mol.

The commercial products comprising silicones of structure (IV) or (V) may include in their composition one or more other aminosilicones, the structure of which is different from formula (IV) or (V).

A product containing aminosilicones of structure (IV) is sold by the company Wacker under the name Belsil® ADM 652.

A product containing aminosilicones of structure (V) is sold by the company Wacker under the name Fluid WR 1300®. Another product containing aminosilicones of structure (IV) is sold by Wacker under the name Belsil ADM LOG 1®.

When these aminosilicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-mean size of the silicone particles in the emulsion generally ranges from 3 nm to 500 nanometres. Preferably, notably as aminosilicones of formula (V), use is made of microemulsions of which the mean particle size ranges from 5 nm to 60 nm (limits included) and more particularly from 10 nm to 50 nm (limits included). Thus, use may be made according to the invention of the aminosilicone microemulsions of formula (V) sold under the names Finish CT 96 ER or SLM 28020® by the company Wacker.

According to a fourth embodiment, the aminosilicones corresponding to formula (II) are chosen from the silicones of formula (VI) below:

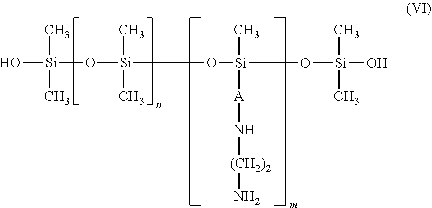

in which:
   m and n are numbers such that the sum (n+m) ranges from 1 to 2000 and in particular from 50 to 150, n denoting a number from 0 to 1999 and notably from 49 to 149, and m denoting a number from 1 to 2000 and notably from 1 to 10; and
   A denotes a linear or branched alkylene radical containing from 4 to 8 carbon atoms and preferably 4 carbon atoms. This radical is preferably linear.

The weight-average molecular mass (Mw) of these aminosilicones preferably ranges from 2000 to 1 000 000 g/mol and more particularly from 3500 to 200 000 g/mol.

A silicone corresponding to this formula is, for example, the Xiameter MEM 8299 Emulsion from Dow Corning.

According to a fifth embodiment, the aminosilicones corresponding to formula (II) are chosen from the silicones of formula (VII) below:

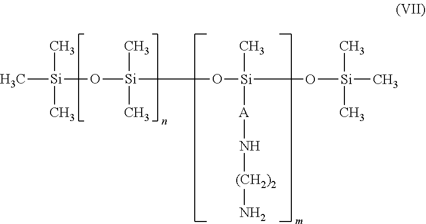

in which:
   m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10; and
   A denotes a linear or branched alkylene radical containing from 4 to 8 carbon atoms and preferably 4 carbon atoms. This radical is preferably branched.

The weight-average molecular mass (Mw) of these aminosilicones preferably ranges from 500 to 1 000 000 g/mol and more particularly from 1000 to 200 000 g/mol.

A silicone corresponding to this formula is, for example, DC2-8566 Amino Fluid from Dow Corning;
c) the aminosilicones corresponding to formula (VIII):

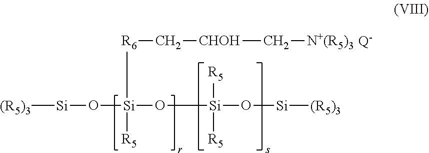

in which:
   R₅ represents a monovalent hydrocarbon-based radical containing from 1 to 18 carbon atoms, and in particular a C₁-C₁₈ alkyl or C₂-C₁₈ alkenyl, for example methyl, radical;
   R₆ represents a divalent hydrocarbon radical, notably a C₁-C₁₈ alkylene radical or a divalent C₁-C₁₈, for example C₁-C₈, alkyleneoxy radical connected to the Si via an SiC bond;
   Q⁻ is an anion such as a halide ion, notably chloride, or an organic acid salt, notably acetate;
   r represents a mean statistical value ranging from 2 to 20 and in particular from 2 to 8; and
   s represents a mean statistical value ranging from 20 to 200 and in particular from 20 to 50.

Such aminosilicones are notably described in patent U.S. Pat. No. 4,185,087.
d) the quaternary ammonium silicones of formula (IX):

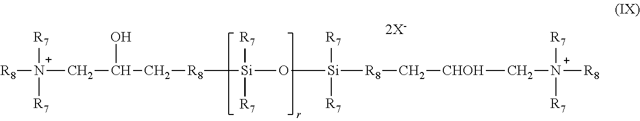

in which:
   R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring comprising 5 or 6 carbon atoms, for example methyl;
   R₆ represents a divalent hydrocarbon radical, notably a C₁-C₁₈ alkylene radical or a divalent C₁-C₁₈, for example C₁-C₈, alkyleneoxy radical connected to the Si via an SiC bond;
   R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl substituent or a —R₆—NHCOR₇ radical;
   X⁻ is an anion such as a halide ion, notably chloride, or an organic acid salt, notably acetate; and
   r represents a mean statistical value ranging from 2 to 200 and in particular from 5 to 100.

Such aminosilicones are notably described in patent application EP-A 0 530 974.
e) the aminosilicones of formula (X):

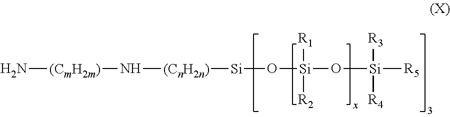

in which:
   R₁, R₂, R₃ and R₄, which may be identical or different, denote a C₁-C₄ alkyl radical or a phenyl group,
   R₅ denotes a C₁-C₄ alkyl radical or a hydroxyl group,
   n is an integer ranging from 1 to 5,
   m is an integer ranging from 1 to 5, and
   x is chosen such that the amine number ranges from 0.01 to 1 meq/g;
f) multiblock polyoxyalkylenated aminosilicones, of the type (AB)ₙ, A being a polysiloxane block and B being a polyoxyalkylenated block including at least one amine group.

Said silicones are preferably formed from repeating units having the following general formulae:

[—(SiMe₂O)ₓSiMe₂—R—N(R″)—R′—O(C₂H₄O)ₐ(C₃H₆O)_{b}—R′—N(H)—R—]

or alternatively

[—(SiMe₂O)ₓSiMe₂—R—N(R″)—R′—O(C₂H₄O)ₐ(C₃H₆O)_{b}—]
in which:
   a is an integer greater than or equal to 1, preferably ranging from 5 to 200 and more particularly ranging from 10 to 100;
   b is an integer between 0 and 200, preferably ranging from 4 to 100 and more particularly between 5 and 30;
   x is an integer ranging from 1 to 10 000 and more particularly from 10 to 5000;
   R″ is a hydrogen atom or a methyl;
   R, which may be identical or different, represent a linear or branched divalent C₂-C₁₂ hydrocarbon-based radical, optionally including one or more heteroatoms such as oxygen; preferably, R, which may be identical or different, denote an ethylene radical, a linear or branched propylene radical, a linear or branched butylene radical or a CH₂CH₂CH₂OCH₂CH (OH) CH₂-radical; preferentially, R denote a CH₂CH₂CH₂OCH₂CH (OH) CH₂-radical; and
   R′, which may be identical or different, represent a linear or branched divalent C₂-C₁₂ hydrocarbon-based radical, optionally including one or more heteroatoms such as oxygen; preferably, R′, which may be identical or different, denote an ethylene radical, a linear or branched propylene radical, a linear or branched butylene radical or a CH₂CH₂CH₂OCH₂CH (OH)CH₂— radical; preferentially, R′ denote —CH(CH₃)—CH₂—.

The siloxane blocks preferably represent 50 mol % and 95 mol % of the total weight of the silicone, more particularly from 70 mol % to 85 mol %.

The amine content is preferably between 0.02 and 0.5 meq/g of copolymer in a 30% solution in dipropylene glycol, more particularly between 0.05 and 0.2.

The weight-average molecular mass (Mw) of the silicone is preferably between 5000 and 1 000 000 g/mol and more particularly between 10 000 and 200 000 g/mol.

Mention may notably be made of the silicones sold under the name Silsoft A-843 or Silsoft A+ by Momentive.
g) the aminosilicones of formulae (XI) and (XII):

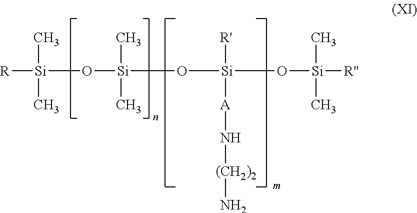

in which:
   —R, R′ and R″, which may be identical or different, denote a C₁-C₄ alkyl group or a hydroxyl group,
   A denotes a C₃ alkylene radical; and
   m and n are numbers such that the weight-average molecular mass of the compound is between 5000 and 500 000

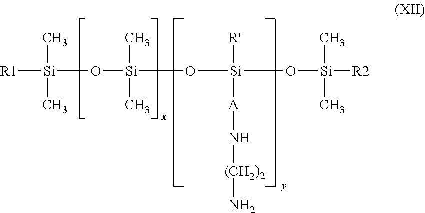

in which:
   x and y are numbers ranging from 1 to 5000; preferably, x ranges from 10 to 2000 and more preferentially from 100 to 1000; preferably, y ranges from 1 to 100;
   R₁ and R₂, which may be identical or different, preferably identical, denote a linear or branched, saturated or unsaturated alkyl group comprising from 6 to 30 carbon atoms, preferably from 8 to 24 carbon atoms and more preferentially from 12 to 20 carbon atoms; and
   A denotes a linear or branched alkylene radical containing from 2 to 8 carbon atoms.

Preferably, A comprises from 3 to 6 carbon atoms, more preferentially 4 carbon atoms; preferably, A is branched.

Mention may be made in particular of the following divalent groups:
—CH₂CH₂CH₂— and —CH₂CH (CH₃) CH₂—.

Preferably, R₁ and R₂ are independent saturated linear alkyl groups comprising 6 to 30 carbon atoms, preferably 8 to 24 carbon atoms and in particular from 12 to 20 carbon atoms; mention may be made in particular of dodecyl, tetradecyl, pentadecyl, hexadecyl, heptadecyl, octadecyl, nonadecyl and eicosyl groups; and preferentially, R₁ and R₂, which may be identical or different, are chosen from hexadecyl (cetyl) and octadecyl (stearyl) groups.

The aminosilicone(s) are preferably of formula (XII) with:
x ranging from 10 to 2000 and in particular from 100 to 1000;
y ranging from 1 to 100;
A comprising from 3 to 6 carbon atoms and notably 4 carbon atoms; preferably, A is branched; more particularly, A is chosen from the following divalent groups:—CH₂CH₂CH₂-and-CH₂CH (CH₃) CH₂—; and
R₁ and R₂ independently being saturated linear alkyl groups comprising from 6 to 30 carbon atoms, preferably from 8 to 24 carbon atoms and in particular from 12 to 20 carbon atoms; chosen notably from dodecyl, tetradecyl, pentadecyl, hexadecyl, heptadecyl, octadecyl, nonadecyl and eicosyl groups; preferentially, R₁ and R₂, which may be identical or different, are chosen from hexadecyl (cetyl) and octadecyl (stearyl) groups.

A silicone of formula (XII) that is preferred is bis-cetearyl amodimethicone. Mention may be made in particular of the aminosilicone sold under the name Silsoft AX by Momentive.
h) polysiloxanes and notably polydimethylsiloxanes, including primary amine groups at only one chain end or on side chains, such as those of formula (XIV), (XV) or (XVI):

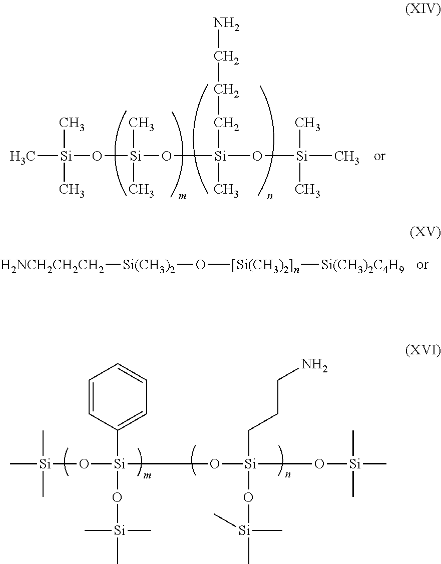

In formula (XIV), the values of n and m are such that the weight-average molecular mass of the aminosilicone is between 1000 and 55 000.

As examples of aminosilicones of formula (XIV), mention may be made of the products sold under the names AMS-132, AMS-152, AMS-162, AMS-163, AMS-191 and AMS-1203 by the company Gelest and KF-8015 by the company Shin-Etsu.

In formula (XV), the values of n and m are such that the weight-average molecular mass of the aminosilicone is between 500 and 3000.

As examples of aminosilicones of formula (XV), mention may be made of the products sold under the names MCR-A11 and MCR-A12 by the company Gelest.

In formula (XVI), the values of n and m are such that the weight-average molecular mass of the aminosilicone is between 500 and 50 000.

As examples of aminosilicones of formula (XVI), mention may be made of the aminopropyl phenyl trimethicone sold under the name DC 2-2078 Fluid by the company Dow Corning.
i) and mixtures thereof.

Preferably, the aminosilicone(s) are chosen from the aminosilicones of formulae (II), (XII) and (XVI) and mixtures thereof, more preferentially chosen from the aminosilicones of formula (XII), and mixtures thereof, and better still the aminosilicone (b) is bis-cetearyl amodimethicone.

Preferably, the additional fatty substance(s), different from the fatty acid triglycerides (ii) containing from 6 to 16 carbon atoms, are chosen from fatty alcohols, fatty esters, plant oils of triglyceride type different from the fatty acid triglycerides (ii), aminosilicones and mixtures thereof.

The total amount of the additional fatty substance(s), different from the fatty acid triglycerides (ii) containing from 6 to 16 carbon atoms, when they are present in the composition according to the invention, preferably ranges from 0.5 to 20% by weight, and more preferentially from 1 to 15% by weight, more preferably from 1.5 to 10% by weight relative to the total weight of the composition.

The total amount of fatty substances (i.e. the sum of the total amount of the fatty acid triglyceride(s) (ii) containing from 6 to 16 carbon atoms and the total amount of the optional additional fatty substance(s)), present in the composition according to the present invention, preferably ranges from 1 to 35% by weight, and more preferentially from 5 to 25% by weight, relative to the total weight of the composition.

The weight ratio (Rb) between the total amount of the additional fatty substance(s), difference from the fatty acid triglycerides (ii) containing from 6 to 16 carbon atoms, and the total amount of the fatty acid triglycerides (ii) containing from 6 to 16 carbon atoms is preferably greater than or equal to 1, more preferentially greater than or equal to 1.5, and better still greater than or equal to 2.

### Cationic Surfactants

The composition according to the present invention may further comprise one or more cationic surfactant(s).

The term “cationic surfactant” means a surfactant that is positively charged when it is contained in the composition according to the invention. This surfactant may bear one or more positive permanent charges or may contain one or more cationizable functions in the composition according to the invention.

The cationic surfactant(s) are preferably chosen from primary, secondary or tertiary fatty amines, which are optionally polyoxyalkylenated, or salts thereof, and quaternary ammonium salts, and mixtures thereof.

The fatty amines generally comprise at least one C₈ to C₃₀ hydrocarbon-based chain.

The cationic surfactant(s) that may be used in the composition of the present invention are preferably chosen from the following quaternary ammonium salts and mixtures thereof:
the quaternary ammonium salts corresponding to the general formula (XVII) below:

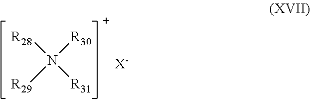

in which the groups R₂₈ to R₃₁, which may be identical or different, represent a linear or branched aliphatic group comprising from 1 to 30 carbon atoms, or an aromatic group such as aryl or alkylaryl, at least one of the groups R₂₈ to R₃₁ denoting a group comprising from 8 to 30 carbon atoms, preferably from 12 to 24 carbon atoms, the aliphatic groups may comprise heteroatoms especially such as oxygen, nitrogen, sulfur and halogens. The aliphatic groups are chosen, for example, from C₁ to C₃₀ alkyl, C₁ to C₃₀ alkoxy, polyoxy (C₂-C₆) alkylene, C₁ to C₃₀ alkylamide, (C₁₂-C₂₂) alkylamido (C₂-C₆) alkyl, (C₁₂-C₂₂) alkyl acetate, and C₁ to C₃₀ hydroxyalkyl groups; X is an anion chosen from the group of halides, phosphates, acetates, lactates, (C₁-C₄) alkyl sulfates, and (C₁-C₄)alkyl- or (C₁-C₄)alkylarylsulfonates.

Among the quaternary ammonium salts of formula (XVII), the ones that are preferred are, on the one hand, tetraalkylammonium salts, for instance dialkyldimethylammonium or alkyltrimethylammonium salts in which the alkyl group comprising approximately from 12 to 22 carbon atoms, in particular behenyltrimethylammonium, distearyldimethylammonium, cetyltrimethylammonium or benzyldimethylstearylammonium salts, or, on the other hand, the palmitylamidopropyltrimethylammonium salt, the stearamidopropyltrimethylammonium salt, the stearamidopropyldimethylcetearylammonium salts, or the stearamidopropyldimethyl (myristyl acetate) ammonium salts sold under the name Ceraphyl® 70 by the company Van Dyk. It is preferred in particular to use the chloride salts of these compounds;
the quaternary ammonium salts of imidazoline, for instance those of formula (XVIII) below:

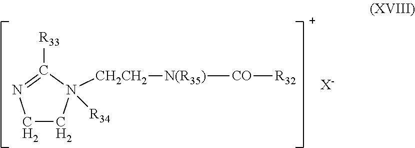

in which:
—R₃₂ represents an alkenyl or alkyl group comprising from 8 to 30 carbon atoms, for example derivatives of tallow fatty acids,
R₃₃ represents a hydrogen atom, a C₁ to C₄ alkyl group or an alkenyl or alkyl group comprising from 8 to 30 carbon atoms,
R₃₄ represents a C₁ to C₄ alkyl group,
R₃₅ represents a hydrogen atom or a C₁ to C₄ alkyl group,
X⁻ is an anion chosen from the group of halides, phosphates, acetates, lactates, alkyl sulfates and alkyl- or alkylarylsulfonates, the alkyl and aryl groups of which preferably comprise, respectively, from 1 to 20 carbon atoms and from 6 to 30 carbon atoms.

Preferably, R₃₂ and R₃₃ denote a mixture of alkenyl or alkyl groups comprising from 12 to 21 carbon atoms, for example derivatives of tallow fatty acids, R₃₄ denotes a methyl group and R₃₅ denotes a hydrogen atom. Such a product is sold, for example, under the name Rewoquat® W 75 by the company Rewo;
the di- or tri-quaternary ammonium salts, in particular of formula (XIX):

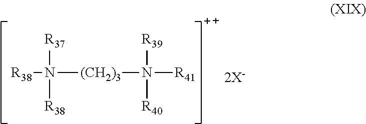

in which:
R₃₆ denotes an alkyl group comprising approximately from 16 to 30 carbon atoms, which is optionally hydroxylated and/or interrupted with one or more oxygen atoms;
R₃₇ is chosen from hydrogen, an alkyl radical comprising from 1 to 4 carbon atoms or an (R₃₆ₐ) (R₃₇ₐ) (R₃₈ₐ)N—(CH₂) 3 group,
R₃₆ₐ, R₃₇ₐ, R₃₈ₐ, R₃₈, R₃₉, R₄₀ and R₄₁, which may be identical or different, are chosen from hydrogen and an alkyl radical comprising from 1 to 4 carbon atoms, and
X⁻ is an anion chosen from the group of halides, acetates, phosphates, nitrates and methyl sulfates.

Such compounds are, for example, Finquat CT-P, sold by the company Finetex (Quaternium 89), and Finquat CT, sold by the company Finetex (Quaternium 75);
the quaternary ammonium salts containing at least one ester function, such as those of formula (XX) below:

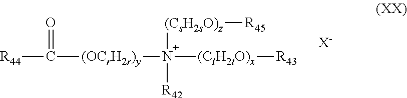

in which:
R₄₂ is chosen from C₁ to C₆ alkyl groups and C₁ to C₆ hydroxyalkyl or dihydroxyalkyl groups;
R₄₃ is chosen from:
   the group

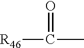

groups R₄₇, which are linear or branched, saturated or unsaturated C₁ to C₂₂ hydrocarbon-based groups,
a hydrogen atom;
R₄₅ is chosen from:
   the group

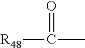

groups R₄₉, which are linear or branched, saturated or unsaturated C₁ to C₆ hydrocarbon-based groups,
a hydrogen atom;
R₄₄, R₄₆ and R₄₈, which may be identical or different, are chosen from linear or branched, saturated or unsaturated C₇ to C₂₁ hydrocarbon-based groups;
r, s and t, which may be identical or different, are integers ranging from 2 to 6;
y is an integer ranging from 1 to 10;
x and z, which may be identical or different, are integers ranging from 0 to 10;
X⁻ is a simple or complex and organic or inorganic anion;

with the proviso that the sum x+y+z is from 1 to 15, that when x is 0 then R₄₃ denotes R₄₇, and that when z is 0 then R₄₅ denotes R₄₉.

The alkyl groups R₄₂ may be linear or branched, and more particularly linear.

Preferably, R₄₂ denotes a methyl, ethyl, hydroxyethyl or dihydroxypropyl group, and more particularly a methyl or ethyl group.

Advantageously, the sum x+y+z ranges from 1 to 10.

When R₄₃ is a hydrocarbon-based group R₄₇, it may be long and contain from 12 to 22 carbon atoms, or may be short and contain from 1 to 3 carbon atoms.

When R₄₅ is a hydrocarbon-based group R₄₉, it preferably contains 1 to 3 carbon atoms.

Advantageously, R₄₄, R₄₆ and R₄₈, which may be identical or different, are chosen from linear or branched, saturated or unsaturated C₁₁ to C₂₁ hydrocarbon-based groups, and more particularly from linear or branched, saturated or unsaturated Cui to C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion that is compatible with the ammonium bearing an ester function.

The anion X⁻ is even more particularly chloride or methyl sulfate.

Use is made more particularly in the dye composition according to the invention of the ammonium salts of formula (XX) in which:
R₄₂ denotes a methyl or ethyl group;
x and y are equal to 1;
z is equal to 0 or 1;
r, s and t are equal to 2;
R₄₃ is chosen from:
   the group

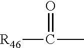

methyl, ethyl or C₁₄ to C₂₂ hydrocarbon-based groups,
a hydrogen atom;
R₄₅ is chosen from:

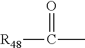

the group
a hydrogen atom;
R₄₄, R₄₆ and R₄₈, which may be identical or different, are chosen from saturated or unsaturated and linear or branched C₁₃ to C₁₇ hydrocarbon-based groups and preferably from saturated or unsaturated and linear or branched C₁₃ to C₁₇ alkyl and alkenyl groups.

Advantageously, the hydrocarbon-based groups are linear.

Examples that may be mentioned include the compounds of formula (XX) such as the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulfate in particular), and mixtures thereof. The acyl groups preferably contain 14 to 18 carbon atoms and are obtained more particularly from a plant oil such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with C₁₀ to C₃₀ fatty acids or with mixtures of C₁₀ to C₃₀ fatty acids of plant or animal origin, or by transesterification of their methyl esters. This esterification is followed by a quaternization using an alkylating agent such as an alkyl halide (preferably a methyl or ethyl halide), a dialkyl sulfate (preferably a methyl or ethyl sulfate), methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

Among these compounds, mention may be made especially of dipalmitoylethylhydroxyethylmethylammonium methosulfate.

The composition according to the present invention may contain, for example, a mixture of quaternary ammonium monoester, diester and triester salts with a weight majority of diester salts.

Use may also be made of the ammonium salts containing at least one ester functional group that are described in patents U.S. Pat. Nos. 4,874,554 and 4,137,180.

Use may be made of behenoylhydroxypropyltrimethylammonium chloride sold by KAO under the name Quatarmin BTC 131.

Preferably, the ammonium salts containing at least one ester function contain two ester functions.

Among the quaternary ammonium salts containing at least one ester function, which may be used, it is preferred to use dipalmitoylethylhydroxyethylmethyl-ammonium salts.

The cationic surfactants that may be present in the composition of the invention are preferably chosen from those of formula (XVII) and mixtures thereof, and more preferentially from behenyltrimethylammonium chloride and cetrimonium chloride.

The total amount of the cationic surfactant(s), when they are present in the composition according to the invention, preferably ranges from 0.5 to 15% by weight, and more preferentially from 1 to 10% by weight, relative to the total weight of the composition.

The weight ratio (Rc) between the total amount of the fatty acid triglycerides (ii) containing from 6 to 16 carbon atoms and the total amount of cationic surfactant(s), when they are present in the composition according to the invention, is preferably greater than or equal to 1, more preferentially this weigh ratio (Rc) ranges from 1 to 10, and better still from 1 to 5.

### Organic Solvents

The composition according to the present invention may further comprise one or more organic solvent(s).

Preferably, the organic solvent(s) are chosen from linear or branched monoalcohols having from 1 to 8 carbon atoms and more preferentially from 1 to 4 carbon atoms, polyols, polyethylene glycols, aromatic alcohols and their mixtures.

Mention may in particular be made, as examples of organic solvents which can be used according to the invention, of ethanol, propanol, butanol, isopropanol, isobutanol, propylene glycol, dipropylene glycol, isoprene glycol, butylene glycol, pentylene glycol, hexylene glycol, glycerol, sorbitol, benzyl alcohol, phenoxyethanol and their mixtures.

The total amount of organic solvent(s), when they are present in the composition according to the invention, preferably ranges from 0.005 to 1% by weight, relative to the total weight of the composition.

### Water

The composition according to the present invention may further comprise water.

The total amount of water, which may be present in the composition according to the present invention, preferably ranges from 50 to 95% by weight, more preferentially from 55 to 95% by weight, and better still from 60 to 80% by weight, relative to the total weight of the composition.

### Additives

The composition according to the present invention may further comprise one or more additive(s) other than the compounds of the invention.

As additives that may be used in accordance with the invention, mention may be made of anionic, non-ionic or amphoteric surfactants, cationic, amphoteric, anionic or non-ionic polymers, antidandruff agents, anti-seborrhoea agents, agents for preventing hair loss and/or for promoting hair regrowth, vitamins and provitamins others than tocopherol, sunscreens, mineral or organic pigments, sequestrants, plasticizers, solubilizers, mineral or organic thickeners, especially polymeric thickeners, opacifiers or nacreous agents, antioxidants, hydroxy acids, fragrances and preserving agents.

Needless to say, a person skilled in the art will take care to select this or these optional additional compound(s) such that the advantageous properties intrinsically associated with the composition according to the invention are not, or are not substantially, adversely affected by the envisaged addition(s).

The above additives may generally be present in an amount, for each of them, of between 0.001% and 20% by weight, relative to the total weight of the composition.

The pH of the composition according to the invention ranges from 7.5 to 10, preferably from 8 to 9.5, more preferably from 8.5 to 9.

The pH of the composition can be adjusted to the desired value by means of commonly used alkalizing agents or acidifying agents. Examples of alkalizing agents are ammonia, alkanolamines, mineral or organic hydroxides. Examples of acidifying agents are mineral or organic acids such as hydrochloric acid, orthophosphoric acid, sulphuric acid, carboxylic acids such as acetic acid, tartaric acid, citric acid, lactic acid and sulphonic acids.

According to a preferred embodiment, the composition of the present invention further comprises one or more alkaline agents.

Among the alkaline agents that can used in the present invention, mention may be made of aqueous ammonia, alkali metal, alkaline earth metal or guanidine (bi) carbonates, alkanolamines, such as mono-, di- and triethanolamines and also derivatives thereof, alkali metal or alkaline earth metal silicates, alkali metal or alkaline earth metal hydroxides, such as sodium or potassium hydroxide, and compounds of the following formula (XXI):

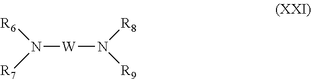

in which formula (XXI) W is a linear or branched (C₁-C₆) alkylene group, in particular propylene, optionally substituted with one or more hydroxyl groups; R₆, R₇, R₈ and R₉, which may be identical or different, represent a hydrogen atom or a C₁-C₄ alkyl radical or a C₁-C₄ hydroxyalkyl radical.

The organic alkaline agent(s) are preferably chosen from organic amines with a pK_{b} at 25° C. of less than 12, preferably of less than 10 and more advantageously still of less than 6. It should be noted that this is the pK_{b} corresponding to the function having the highest basicity. In addition, the organic amines do not comprise any alkyl or alkenyl fatty chain comprising more than ten carbon atoms.

Preferably, the alkaline agent(s) are chosen from aqueous ammonia, alkanolamines such as monoethanolamine or ethanolamine, carbonates, especially guanidine carbonate, silicates, such as sodium silicate, and mixtures thereof.

Another object of the present invention relates to a cosmetic process for styling and/or conditioning keratin fibres, in particular human keratin fibres such as hair, which consists in applying to said keratin fibres a composition as previously defined, and after an optional leave-on time, optionally removing it by rinsing.

Preferably, the present invention relates to a cosmetic process for combing and/or styling keratin fibers, in particular human keratin fibers such as hair, which consists in applying to said keratin fibers a composition as previously defined, and after an optional leave-on time, optionally removing it by rinsing.

The composition can be used as a leave-on product.

After an optional leave-on time, the composition may preferably be removed by rinsing with water. The fibres may then be dried or left to dry.

Generally, the leave-on time of the composition on the keratin fibres may range from a few seconds to 12 hours, preferably from 5 seconds to 60 minutes and even better still from 10 seconds to 15 minutes, or more better from 10 seconds to 5 minutes.

The composition may be applied to wet or dry keratin fibres, preferably wet keratin fibres.

The composition according to the invention is generally applied to dry keratinous substances which have optionally been washed with a shampoo.

In the present invention, the term “keratin fibres” denotes human keratin fibres, and in particular human hair.

The example that follows serves to illustrate the invention without, however, being limiting in nature.

### EXAMPLES

In the example that follow and unless otherwise indicated, the amounts are given as mass percentages of active material (am) relative to the total weight of the composition.

### I. Risk of Breakage During Combing

a. Compositions

The following compositions A1 (invention) and B1 and B2 (comparative) were prepared from the ingredients indicated in table 1 below (w % of active material (AM)):

TABLE 1

| | A1 | B1 | B2 |
|---|---|---|---|
| | (invention) | (comparative) | (comparative) |
| Cocos nucifera | 5 | — | 5 |
| (coconut) oil | | | |
| Cysteine HCl | 0.75 | 0.75 | — |
| Cetearyl alcohol | 5 | 5 | 5 |
| Isononyl isononanoate | 2.5 | 2.5 | 2.5 |
| Behentrimonium | 3.95 | 3.95 | 3.95 |
| chloride (VARISOFT | | | |
| BT 85 - EVONIK) | | | |
| Cetyl esters (and) cetyl | 2.5 | 2.5 | 2.5 |
| esters (CRODAMOL | | | |
| MS-PA-(MH) - | | | |
| CRODA) | | | |
| Prunus armeniana | 0.25 | 0.25 | 0.25 |
| (apricot) seed powder | | | |
| Bis-cetearyl | 0.4 | 0.4 | 0.4 |
| amodimethicone | | | |
| (SILSOFT AX-E PMF- | | | |
| MOMENTIVE | | | |
| PERFORMANCE | | | |
| MATERIALS) | | | |
| Ethanolamine | 0.53 | 0.53 | 0.53 |
| Hydroxyethylcellulose | 0.4 | 0.4 | 0.4 |
| Tocopherol | 0.1 | 0.1 | 0.1 |
| Fragrance | 1 | 1 | 1 |
| pH agent | Qs pH 8.8 | Qs pH 8.8 | Qs pH 8.8 |
| Water | Qs 100 | Qs 100 | Qs 100 |

pH of the formula = 8.8

b. Protocol

In this mechanical evaluation method, hair fibers are subjected to repeated low-amplitude extension cycles until either the fiber breaks or the maximum number of cycles set by the operator is reached. At the end of the fatigue test, the lifetime of the fiber is measured. This corresponds to the number of extension cycles that the fiber has withstood without breaking.

The mechanical tensile stress that is applied to the fiber can be constant deformation, constant force or constant stress. For this analysis, cyclic fatigue tests were performed at a constant stress of 144 MPa, using a Diastron CYC 801 cyclic fatigue tester. Cross sectional area measurements are carried out using a Fiber Dimensional Analysis System (FDAS, Diastron) and are used to achieve correct targeted stress.

The following protocol was used for each composition A1, B1 and B2 on Virgin multi ethnic hair (Curl pattern 4-5) from IHIP:
1. Prepare 1 g hair swatch
2. Wash 1 g hair with 0.4 g Au Naturale shampoo (commercial product)
3. Towel dry swatch
4. Apply 4 g of each composition A1, B1 and B2 to 1 g damp hair swatch
5. Leave treated hair on hot plate at 31 Degrees Celsius (for 15 minutes)
6. Rinse treatment off after 15 minutes
7. Blow Dry hair using cold air, and
8. Comb hair (120 strokes)

Steps 2-8 of the above protocol have been repeated 5 times (5 treatments and 600 strokes)

The following protocol was used as control on Virgin multi ethnic hair (Curl pattern 4-5) from IHIP:
1. Prepare 1 g hair swatch
2. Wash 1 g hair with 0.4 g Au Naturale shampoo
3. Towel dry swatch
4. Blow Dry hair using cold air, and
5. Comb hair (120 strokes).

Steps 2-5 of the above control protocol have been repeated 5 times (5 treatments and 600 strokes).

c. Results

The results thus obtained for each composition A1, B1 and B2 are expressed in table 2 here below. The more cycles before breakage, the stronger is the hair.

TABLE 2

| | A1 | B1 | B2 | |
|---|---|---|---|---|
| | (invention) | (comparative) | (comparative) | Control |
| Average cycle | 6560 | 5154 | 4249 | 4333 |
| before failure | | | | |

The results thus obtained show that the claimed composition A1, which comprises the combination of cysteine with a particular fatty acid triglyceride, permits to reinforce curly hair through the increase in average number of cycles before failure. Indeed, the number of cycles before failure is significantly higher when hair is treated with the claimed composition A1 compared to the comparative compositions B1 and B2 comprising either cysteine or the particular fatty acid triglyceride only.

Thus, the combination of cysteine with at least one fatty acid triglyceride containing from 6 to 16 carbon atoms provides a synergetic effect regarding the reinforcement of curly hair.

### II. Ease of Combing

a. Compositions

Compositions A1 (invention) and B1 and B2 (comparative) were prepared from the ingredients indicated in table 1 of example I.

b. Protocol

The combing method measures the force of combing a swatch on a Lloyd extensiometer (Ametek Inc, USA). The lower the force is, the easier is to comb the hair.

A wide tooth comb accessory, resembling a typical comb used for natural African hair was used. The afro comb had teeth ˜3 mm apart and was secured on the instrument gantry as shown below. To carry out the test, a swatch (˜1 g) is inserted at the center of the comb and the swatch is clamped to the sensor. The swatch is moved by the gantry in a linear translation and the combing forces are measured. This movement is repeated 4 times and the average combing force is used to assess efficacy of the tested composition. The lower the force is, the easier is to comb the hair.

The following protocol was used for each composition A1, B1 and B2 on Curl pattern 7-8 hair from South Africa:
1. Each 1 g swatch of afro hair was washed with 0.4 g Au Naturale shampoo (commercial product) then rinsed.
2. 1 g of each composition A1, B1 and B2 was applied to each gram of damp hair from step 1.
3. Leave treated hair on heating plate at 31 Degrees Celsius (for 15 minutes)
4. The swatch was then rinsed and left to dry for 24 hours at room temperature (±22° C.).

Steps 1-4 of the above protocol have been repeated 5 times.

A similar protocol comprising only steps 1, 3 and 4 of the above protocol was used as control.

At the end of the process, the hair swatches were left to air dry for 24 hours then characterized for the force of combing using a Lloyd tensiometer.

c. Results

The results thus obtained for each composition A1, B1 and B2 are expressed in table 3 here below.

TABLE 3

| | A1 | B1 | B2 | |
|---|---|---|---|---|
| | (invention) | (comparative) | (comparative) | Control |
| Total work | 1472 | 2246 | 1917 | 2283 |
| required to | | | | |
| comb the hair | | | | |
| (mean in | | | | |
| Joules) | | | | |

The results thus obtained show that the claimed composition A1, which comprises the combination of cysteine with a particular fatty acid triglyceride improves the combing of very curly hair. Indeed, the combing is significantly easier when hair is treated with the claimed composition A1 compared to the comparative compositions B1 and B2 comprising either cysteine or the particular fatty acid triglyceride only.

Thus, the combination of cysteine with at least one fatty acid triglyceride containing from 6 to 16 carbon atoms provides a synergetic effect regarding the ease of combing of very curly hair.

### III. Hair Strengthening

a. Composition

The following composition A2 (invention) was prepared from the ingredients indicated in table 4 below (w % of active material (AM)):

TABLE 4

| | A2 |
|---|---|
| Cocos nucifera | 5 |
| (coconut) oil | |
| Cysteine HCl | 0.75 |
| Cetearyl alcohol | 5 |
| Isononyl isononanoate | 2.5 |
| Behentrimonium | 3.95 |
| chloride (VARISOFT | |
| BT 85 -EVONIK) | |
| Cetyl esters (and) cetyl | 2.5 |
| esters (CRODAMOL | |
| MS-PA-(MH) - | |
| CRODA) | |
| Prunus armeniana | 0.25 |
| (apricot) seed powder | |
| Bis-cetearyl | 0.4 |
| amodimethicone | |
| (SILSOFT AX-E PMF- | |
| MOMENTIVE | |
| PERFORMANCE | |
| MATERIALS) | |
| Ethanolamine | 0.6 |
| Hydroxyethylcellulose | 0.4 |
| Tocopherol | 0.1 |
| Ascorbic Acid | 0.2 |
| Sodium Metabisulfite | 0.2 |
| Fragrance | 1 |
| pH agent | Qs pH 8.8 |
| Water | Qs 100 |

pH of the formula = 8.8

b. Protocol

In this mechanical evaluation method, the effect of a formula at reducing textured hair fiber breakage is measured by a wet repeated grooming technique. To evaluate the breakage, the method is to hold the tress in one hand over a paper sheet and then comb it 100 times with a rat-tail comb. The strokes should pass quickly through the entire length of the tress, otherwise the comb will remain stuck in the hair.

The hair is sprayed with a water mist every 20 strokes so that tress will not dry out (6-inches distance, spray a mist of water 3× each side of tress).

The broken fibers on the paper sheet and in the comb are then counted. The average control breakage should not be lower than 50 fibers.

The following protocol was used for the composition A2 on Virgin afro hair (Curl pattern 7-8) from Tsebo Technical Services (South Africa):
1. Prepare 0.25 g hair swatch
2. Wash 0.25 g hair with 0.1 g “Dark&Lovely 3in1” shampoo (commercial product)
3. Towel dry swatch
4. Apply 1 g of the composition A2 to 0.25 g damp hair swatch
5. Leave treated hair on hot plate at 31 Degrees Celsius (for 15 minutes)
6. Rinse treatment off after 15 minutes
7. Towel-dry the swatch and ensure that the swatch is damp (not dripping wet).
8. Perform Wet Repeated Grooming

Steps 2-8 of the above protocol have been repeated 5 times (5 treatments)

A similar protocol comprising only steps 1, 2, 3 and 8 of the above protocol was used as control.

c. Results

The results thus obtained for the composition A2 are expressed in table 5 here below.

TABLE 5

| | A2 | Control |
|---|---|---|
| Cumulative | 164 | 267 |
| Number of | | |
| Broken Fibers | | |

The results thus obtained show that the claimed composition A2, which comprises the combination of cysteine with a particular fatty acid triglyceride improves the strength of very curly hair. Indeed, the number of broken fibers during wet combing is significantly lower when hair is treated with the claimed composition A2 compared to the control.

### IV. Compositions According to the Present Invention

The following compositions A3, A4 and A5 according to the present invention were prepared from the ingredients indicated in table 6 below (w % of active material (AM)):

TABLE 6

| | A3 | A4 | A5 |
|---|---|---|---|
| Cocos nucifera | 5 | 5 | 5 |
| (coconut) oil | | | |
| Cysteine | — | 0.65 | 0.65 |
| Cysteine HCl | 0.75 | — | — |
| Cetearyl alcohol | 4.5 | 4.5 | 4.5 |
| Isononyl isononanoate | 2.5 | 2.5 | 2.5 |
| Behentrimonium | 3.95 | 3.95 | 3.95 |
| chloride (VARISOFT | | | |
| BT 85 - EVONIK) | | | |
| Cetyl esters (and) cetyl | 2 | — | 2 |
| esters (CRODAMOL | | | |
| MS-PA-(MH) - | | | |
| CRODA) | | | |
| Prunus armeniana | 0.25 | 0.25 | 0.25 |
| (apricot) seed powder | | | |
| Bis-cetearyl | 0.4 | 0.4 | 0.4 |
| amodimethicone | | | |
| (SILSOFT AX-E PMF- | | | |
| MOMENTIVE | | | |
| PERFORMANCE | | | |
| MATERIALS) | | | |
| Ethanolamine (Qs) | Qs pH 9 | Qs pH 9 | Qs pH 9 |
| Hydroxyethylcellulose | 0.4 | — | 0.4 |
| Tocopherol | 0.1 | 0.1 | 0.1 |
| PEG-180 | 2 | 2 | 2 |
| Isopropyl alcohol | — | 0.9 | 0.9 |
| Colorant | — | qs | — |
| Fragrance | 1 | 1 | 0.9 |
| Water | Qs 100 | Qs 100 | Qs 100 |

Hair treated with compositions A3, A4 and A5 thus obtained is not only easy to detangle and fast to comb, but its manageability to style is also very good. Moreover, compositions A3-A4 confer good conditioning properties to the hair, notably in terms of softness and volume. The hair is also strengthened.

1-18. (canceled)

19. A composition comprising:
(i) cysteine, derivatives thereof, and/or salts thereof in a total amount of less than or equal to 1% by weight, relative to the total weight of the composition,
(ii) at least one fatty acid triglyceride comprising from 6 to 16 carbon atoms in a total amount of greater than or equal to 1% by weight, relative to the total weight of the composition, and
(iii) optionally at least one additional reducing agent other than (i) cysteine and/or one of derivatives thereof and salts thereof in a total amount of less than or equal to 1% by weight, relative to the total weight of the composition;
wherein the pH of the composition ranges from 7.5 to 10.

20. The composition of claim 19, wherein the total amount of (i) cysteine, derivatives thereof, and/or salts thereof ranges from 0.05% to 1% by weight, relative to the total weight of the composition.

21. The composition of claim 19, wherein the (ii) at least one fatty acid triglyceride comprises from 6 to 14 carbon atoms.

22. The composition of claim 19, wherein the (ii) at least one fatty acid triglyceride is chosen from coconut oil, caprylic/capric acid triglycerides, palm kernel oil, babasu oil, cuphea oil, or mixtures thereof.

23. The composition of claim 19, wherein the total amount of (ii) fatty acid triglyceride(s) ranges from 1% to 15% by weight, relative to the total weight of the composition.

24. The composition of claim 19, wherein the weight ratio of the total amount of (ii) fatty acid triglyceride(s) to the total amount of (i) cysteine, derivatives thereof, and/or salts thereof is greater than or equal to 1.

25. The composition of claim 19, wherein the (iii) at least one additional reducing agent is chosen from non-thiol reducing agents or mixtures thereof.

26. The composition of claim 19, wherein the (iii) at least one additional reducing agent is chosen from sulfites, sulfite derivatives, ascorbic acid, ascorbic acid derivatives, tocopherols, tocopherol derivatives, or mixtures thereof.

27. The composition of claim 19, wherein the total amount of (iii) additional reducing agents ranges from 0.05% to 1% by weight, relative to the total weight of the composition.

28. The composition of claim 19, wherein the composition is free of (iii) additional reducing agents.

29. The composition of claim 19 further comprising (iv) at least one additional fatty substance different from the (ii) at least one fatty acid triglyceride.

30. The composition of claim 29, wherein the total amount of the (iv) additional fatty substance(s), ranges from 0.5% to 20% by weight, relative to the total weight of the composition.

31. The composition of claim 29, wherein the total amount of fatty substances (ii) and (iv) ranges from 1% to 35% by weight, relative to the total weight of the composition.

32. The composition of claim 19, wherein the weight ratio of the total amount of (iv) additional fatty substance(s) to the total amount of (ii) fatty acid triglycerides is greater than or equal to 1.

33. The composition of claim 19, further comprising one or more cationic surfactant(s).

34. The composition of claim 33, wherein the cationic surfactants are chosen from:
quaternary ammonium salts corresponding to the general formula (XVII): 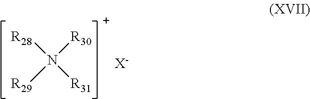
wherein the groups R₂₈ to R₃₁ are independently chosen from a linear or branched aliphatic group comprising from 1 to 30 carbon atoms, or an aromatic group, wherein at least one of the groups R₂₈ to R₃₁ represents a group comprising from 8 to 30 carbon atoms, and the aliphatic groups optionally comprise heteroatoms;
quaternary ammonium salts of imidazoline;
di- or tri-quaternary ammonium salts;
quaternary ammonium salts comprising at least one ester function; or mixtures thereof.

35. The composition of claim 33, wherein the total amount of cationic surfactants ranges from 0.5% to 15% by weight, relative to the total weight of the composition.

36. The composition of claim 33, wherein the weight ratio of the total amount of (ii) fatty acid triglycerides to the total amount of cationic surfactant(s) is greater than or equal to 1.

37. The composition of claim 19, further comprising water in a total amount ranging from 50% to 95% by weight, relative to the total weight of the composition.

38. A method for treating keratin fibers, comprising:
(a) applying to the keratin fibers a composition comprising:
(i) cysteine, derivatives thereof, and/or salts thereofin a total amount of less than or equal to 1% by weight, relative to the total weight of the composition,
(ii) at least one fatty acid triglyceride comprising from 6 to 16 carbon atoms in a total amount of greater than or equal to 1% by weight, relative to the total weight of the composition, and
(iii) optionally at least one additional reducing agent other than (i) cysteine and/or one of derivatives thereof and salts thereof in a total amount of less than or equal to 1% by weight, relative to the total weight of the composition;
wherein the pH of the composition ranges from 7.5 to 10, and
(b) after an optional leave-on time, optionally removing the composition by rinsing.
